(19) United States
(12) Patent Application Publication
(10) Pub. No.: US 2004/0138705 A1
(43) Pub. Date: Jul. 15, 2004

(54) Surgical staple for tissue treatment

(76) Inventors: Harri Heino, Tampere (FI); Kimmo Lahteenkorva, Tampere (FI); Pertti Tormala, Tampere (FI); Gregory C. Fanelli, Danville, PA (US); Juha-Pekka Nuutinen, North Wales, PA (US)

Correspondence Address: KENYON & KENYON, 1500 K STREET, N.W., SUITE 700, WASHINGTON, DC, 20005, US

(21) Appl. No.: 10/338,686

(22) Filed: Jan. 9, 2003

(51) Int. Cl. A61B 17/08

(52) U.S. Cl. 606/219

(57) ABSTRACT

A fastener is provided for tissue fixation. The fastener includes first and second implantation members and a connecting member connecting the first and second implantation members. The implantation members may have protuberances on their surfaces. A method for using the fastener and a kit including the fastener and an installation tool is also provided.

### FIELD OF THE INVENTION

The present invention relates to surgical implants for repairing tissue or attaching matter to tissue. More specifically, the present invention relates to a surgical fastener or device formed in the shape of a staple comprising at least two implantation members connected by a connecting member, where the implantation members have protrusions and preferably pointed ends.

### BACKGROUND OF THE INVENTION

It has been shown that the fixation of meniscus traumas, like ruptures and lesions, by suturing with absorbable sutures gives better results than the removal of traumatized meniscal tissue, see e.g. N. A Palmeri, T. F. Winters, A. E. Joiner and T. Evans, “The Development and Testing of the Arthroscopic Meniscal Staple”, Arthroscopy, Vol. 5, No. 2, 1989, p. 156. However, arthroscopic suturing is a complicated and tedious technique, with significant risks to the patient because of the danger of damaging vessels and nerves. Therefore, surgeons for a long time have wanted an absorbable meniscus lesion fixation device, such as a fastener, which has the advantages of absorbable suturing techniques, but which may be used more rapidly and safely than sutures.

Several research groups have tried to develop such absorbable meniscus lesion fixation devices, however, the demands of such a device are high. For example, it must be strong enough to maintain good contact with the lesion tissues after an operation and retain its strength long enough to allow for rapid healing. The device should also not cause any damage to the cartilage surfaces of the femur and tibia, and it must be absorbed without causing complications that would prevent or hinder the healing of a lesion. Additionally, the installation of the devices should be easy and rapid and should cause minimal operational trauma. Because of these high demands, the optimal absorbable meniscus lesion fixation device has not yet been developed.

Palmeri et al., supra, reported the development of a method of meniscal repair using arthroscopically applied absorbable fasteners or staples. However, the reported method was complicated because the final design used cannulation of the staple for needle-guided placement. Additionally, staple fracture, migration and articular abrasion were found.

U.S. Pat. No. 4,873,976 to Schreiber discloses an arrow-like implant specifically intended for the surgical repair of meniscal ruptures. However, the disclosed arrow-like implant has the disadvantage that its proximal end (stem) may cause tissue irritation and abrasion, particularly when placed in connection with the meniscus, because the stem may be left protruding from the outer surface of the meniscus.

U.S. Pat. No. 4,635,637 to Schreiber describes a surgical suture having a base member, two substantially parallel shafts upstanding from said base member and pointed barbs at the ends thereof. In the described embodiments the base member is as thick as the shafts. However, base thicknesses below 1 mm would be preferable e.g. in meniscus rupture fixation to minimize the risk that the base member could damage the opposite (distal femoral) cartilage surface.

U.S. Pat. Nos. 4,884,572 and 4,895,141 Bays et al. describe a surgical-repair tack and applicator, and a method of using them. The tack has a barb member, a shaft portion and a grip portion. The tack is made of biodegradable material having a degradation time selected to coincide with the healing time of the tissue. In an alternate embodiment, the tack's barb comprises a continuous helix. A disadvantage of this tack is that the grip portion is bulky and may remain on the meniscal surface causing irritation inside a joint cavity.

The method and apparatus for repairing a meniscal tear disclosed by U.S. Pat. No. 5,059,206 to Winters comprises a fastener having protrusions or barbs that is applied to a meniscal tear with a delivery device. The delivery device has a flexible tip that is manipulable through a curved radius. This enables the surgeon to insert the device into the central part of the knee and then extend the fastener radially outward into and across a meniscal tear. However, the proximal end of the fastener including a cylindrical end (head member) is bulky and protrudes partially above and/or below the outer surface of the meniscus.

U.S. Pat. No. 5,562,704 to Tamminmäki et al. discloses an arrow-like bioabsorbable implant particularly intended for the surgical repair of meniscal ruptures. This implant does not have the guiding or abrasion problems that implants of U.S. Pat. No. 4,873,976 or U.S. Pat. No. 5,059,206 may have. However, the disclosed implant only comprises one body including a plurality of cuts (arresting members), therefore a minimum of two such implants would have to be installed to receive a similar fixation effect as one staple (with two shafts).

U.S. Pat. No. 5,569,252 to Justin et al. describes a fastener, an installation device, and a method for repairing tears in the soft tissue of a patient, including meniscal tears. The fastener has a variable-pitch helical protrusion along a central portion that decreases from the distal end to the proximal end. The fastener can serve to bring two sides of the tear into opposition as it is advanced across the two sides of the tear in a screwing motion. This implant, which requires a screwing/turning motion for installation, is slow and tedious to use arthroscopically. In addition, turning the implant through fibrous tissue, such as meniscus tissue, risks the fibrous tissue twisting around the implant, thereby hindering or preventing the installation of the implant, or damaging the tissue.

Patent application PCT/EP 98/04183 describes a fastener for body tissue repair. Although this implant sinks totally inside a tissue, like knee meniscus, the first protrusions can be damaged, bent or broken during the insertion of the implant into tissue. Also the fixation strength of this kind of implant is not as good as that of implants that are located partially on the surface of the meniscus (see e.g. S. P. Arnoczky and M. Lavagnino, Am. J. Sports Med. 29 (2001) 118-123).

EP 0 770 354 A1 to Person et al. describes an apparatus having a series of fasteners and a firing bar located on top of one another on the inside of a support casing. This arrangement causes the support casing to be thick, which may cause difficulties in pushing the support casing into a narrow knee joint for firing fastener(s) into the meniscus tissue.

EP 1070 487 A2 to Bowman describes a graft fixation implant having longitudinal passages through implantation members. Because of these passages, the implantation members must be relatively thick, thereby requiring the formation of large and traumatic drill holes in the tissue.

U.S. Ser. No. 2001/0,029,382 A1 to Bowman and Bruker describe a fixation device comprising implantation members, which are thick because of passages (for mounting prongs) running through them.

Orthopedic Product News (January/February 2002, p.14) describes Arthrotek Staples as resorbable meniscal repair implants. These are U-shaped, two pronged implants. However, the curved part of the implant remaining on the meniscal surface is as thick as the straight, sharp-tip implantable parts of the staple and therefore may cause initiation.

Because of the limitations of prior art implants, a need exists for a bioabsorbable fastener that allows a minimally invasive method for repairing a tear in soft or tough tissue and/or for fixation of synthetic fibrous implants or living tissue transplants on or in living tissue.

A need also exists for such a fastener that is rapid and easy to install and gives a strong and safe fix to the tissue tear, implant or transplant, and is minimally traumatic. The fastener may be made from a non-toxic, biocompatible bioabsorbable polymer, polymer alloy or fiber reinforced polymer composite, specially designed to maintain its structural integrity during the healing of the tear and to prevent tissue abrasion.

A need also exists for a fastener having a shape designed to compress the tear.

A need also exists for a fastener that can penetrate the tissue being repaired (such as a meniscal tear) and hold the ruptured edges together while causing minimal trauma to the tissue through which the fastener travels.

A need also exists for a minimally traumatic fastener, which can be shot or pushed from behind through a thin cannula with a piston into tissue without gripping the device with the piston.

A need also exists for a fastener, which, when shot or pushed from behind through a thin cannula with a piston, penetrates tough tissue, like meniscus tissue, without the need of mounting prongs.

A need also exists for a fastener, which has a thin, strong and tough, but flexible, monofilament-like part (connecting member), connecting thicker implantation members.

A need also exists for a fastener that, once installed, will leave only a small and thin part of the proximal, monofilament-like part of the fastener on the surface of the tissue and does not protrude from the surface of the tissue when the tissue is compressed under load (e.g. during walking).

These and other objects may be attained with the fastener of the present invention.

### SUMMARY OF THE INVENTION

One embodiment of the present invention provides a fastener for tissue repair comprising, a first longitudinal member having distal and proximal ends and transverse protrusions, a second longitudinal member having distal and proximal ends and transverse protrusions, and a connecting member connecting the proximal end of the first member to the proximal end of the second member where at least a portion of the connecting member has a smaller diameter than a portion of either the first member or the second member.

Another embodiment of the present invention provides a method for repairing tissue using the fastener described above, including pushing the distal portions of the first and second longitudinal members of the fastener into the tissue and partially embedding the fastener in the tissue, where at least a portion of the connecting member is visible on the surface of the tissue.

Yet another embodiment of the present invention includes a method of fixating an implant to tissue using the fastener described above, comprising placing the implant on the tissue, pushing the distal portions of the first and second members of the fastener through the implant into the tissue, and embedding the fastener into the tissue, where at least a portion of the connecting member of the fastener is visible on a surface of the implant.

Another embodiment of the present invention includes, a kit comprising, the fastener described above and an insertion tool, comprising a cannula, a piston, and a tip.

In another embodiment of the present invention, a method is provided of using the kit described above, comprising, loading a fastener into an insertion tool, wherein the distal portions of the first member and the second member are proximal to the insertion tool tip, pressing the tip of the insertion tool against a tissue, pushing the fastener into the tissue through the tip of the insertion tool by accelerating the piston and stopping the insertion of the fastener into the tissue through a stopper, wherein at least a portion of the connecting member of the fastener is visible on a surface of the tissue.

Finally, another embodiment of the present invention, provides a method of manufacturing a fastener described above, comprising, extruding a billet of bioabsorbable material, cutting the billet, and bending the cut billet into the form of the fastener.

### BRIEF DESCRIPTION OF THE DRAWINGS

FIGS. 1A-R show perspective views of different embodiments of fasteners according to the present invention.

FIG. 2 shows a perspective view of an embodiment of a ridged fastener in accordance with the present invention.

FIG. 3 shows a cross section of FIG. 2 along the A-A line.

FIGS. 4A-V show different embodiments of ridge profiles of implantation members in accordance with the present invention.

FIGS. 5A-E show cross sections of embodiments of the present invention, which may be either implantation or connecting members.

FIG. 6 shows a Prior Art example of the fibrous structure of meniscus tissue.

FIG. 7 shows another Prior Art example of the fibrous structure of meniscus tissue.

FIGS. 8A-D show an embodiment of a method of the present invention for inserting a fastener of the present invention into a torn meniscus.

FIG. 9 illustrates the orientation of the fibrous structure of a meniscus in relation to an installed fastener of the present invention.

FIG. 10 illustrates, as seen from above, the location of the proximal, monofilament suture-like part of an embodiment of the connecting member of a fastener of the present invention on the surface of a meniscus.

FIG. 11 shows a cross section of an embodiment of a fastener of the present invention closing a tissue tear.

FIG. 12 shows another embodiment of a fastener of the present invention closing a tissue tear.

FIGS. 13A-B show an embodiment of a method of the present invention for closing a wound.

FIGS. 14A-B show a method of separating a tissue using an embodiment of a fastener of the present invention.

FIGS. 15A-B show cross-sections of tissue compressed using an embodiment of the fastener of the present invention.

FIG. 16 shows a top view of the fixation of a fibrous mesh on the surface of living tissue by means of an embodiment of fasteners of the present invention.

FIG. 17 shows a cross section along line B-B of FIG. 16.

FIGS. 18A-D show cross sections of an embodiment of an installation device of the present invention, including two pistons and a method of using the installation device with a fastener of the present invention.

### DETAILED DESCRIPTION

Like numbers will be used for like elements in the Figures.

The present invention provides a bioabsorbable fastener or staple that allows minimally invasive repair of a tear in soft or tough tissue. The fastener of the present invention may also be used to fix synthetic fibrous implants or living tissue transplants on or in tissue. The provided fastener is easy to install by pushing or shooting from behind. The fastener may be made from a non-toxic, biocompatible, bioabsorbable, polymer, polymer alloy or fiber reinforced polymer composite, which maintains its structural integrity during the healing of a tear and prevents tissue abrasion. The shape of the fastener may preferably be that of a staple.

In embodiments of the present invention the fastener may compress a tissue tear or maintain portions of a tissue apart. In another embodiment of the present invention a portion of the fastener remains on the surface of the tissue, forming a monofilament suture-loop like small prominence on the tissue surface.

The present invention may be used to heal a knee meniscal tear, close wounds of connective tissues, affix synthetic hernia meshes or non-woven collagen felts to tissue, and in surgery to repair traumas to soft and/or tough tissues containing fibrous structures.

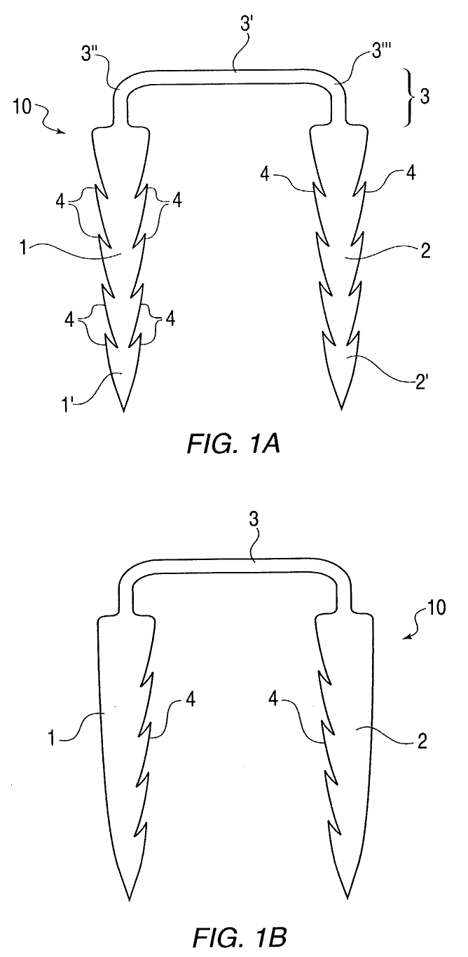

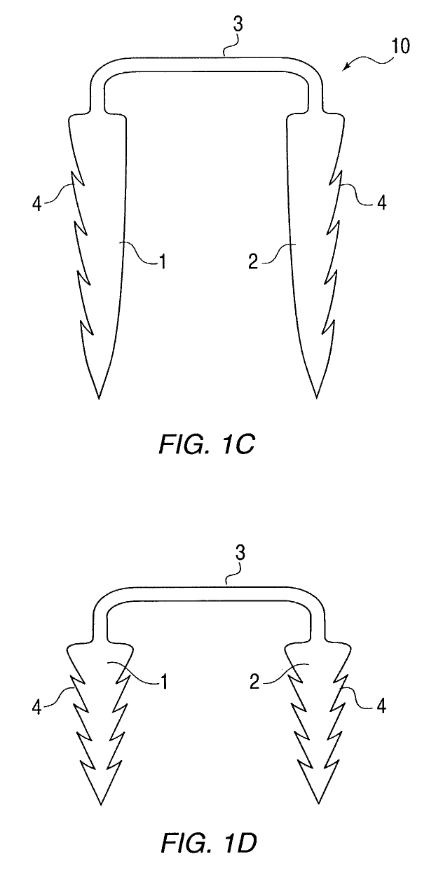

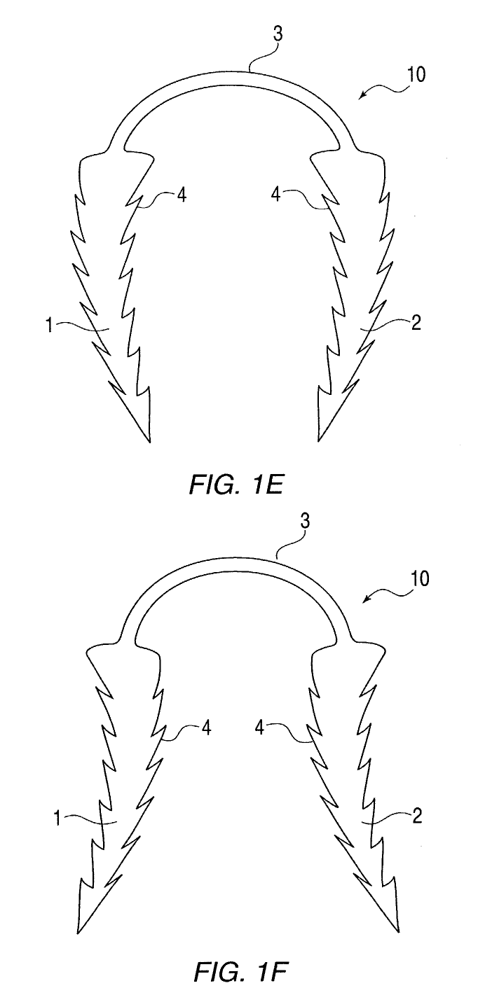

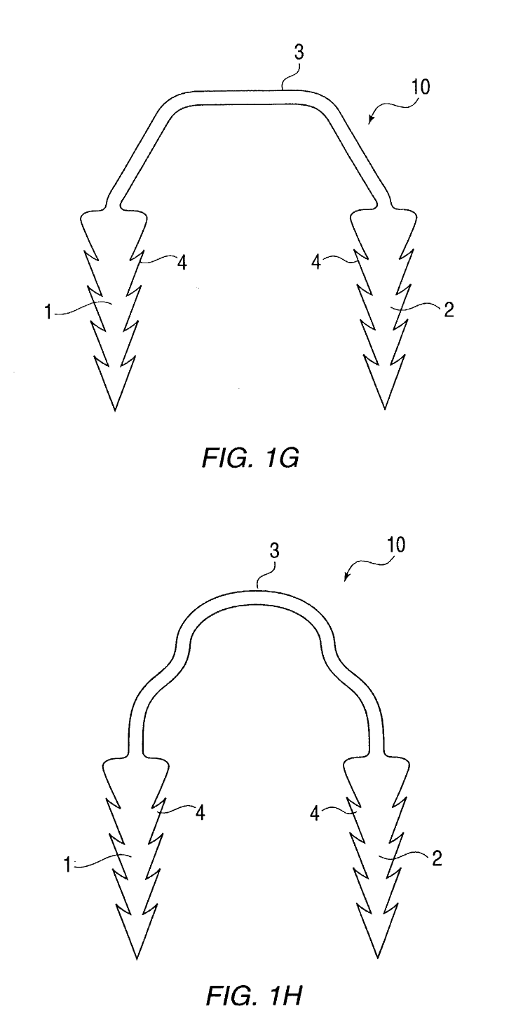

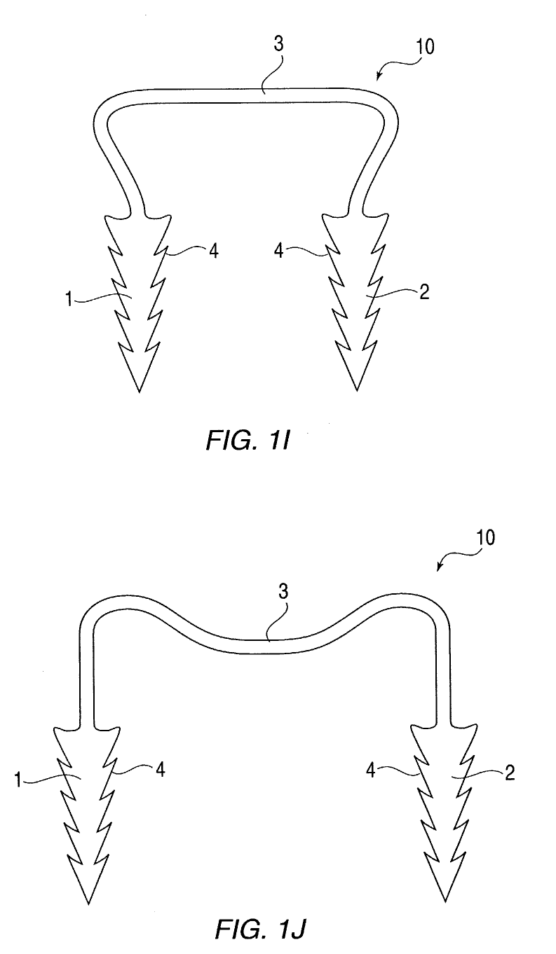

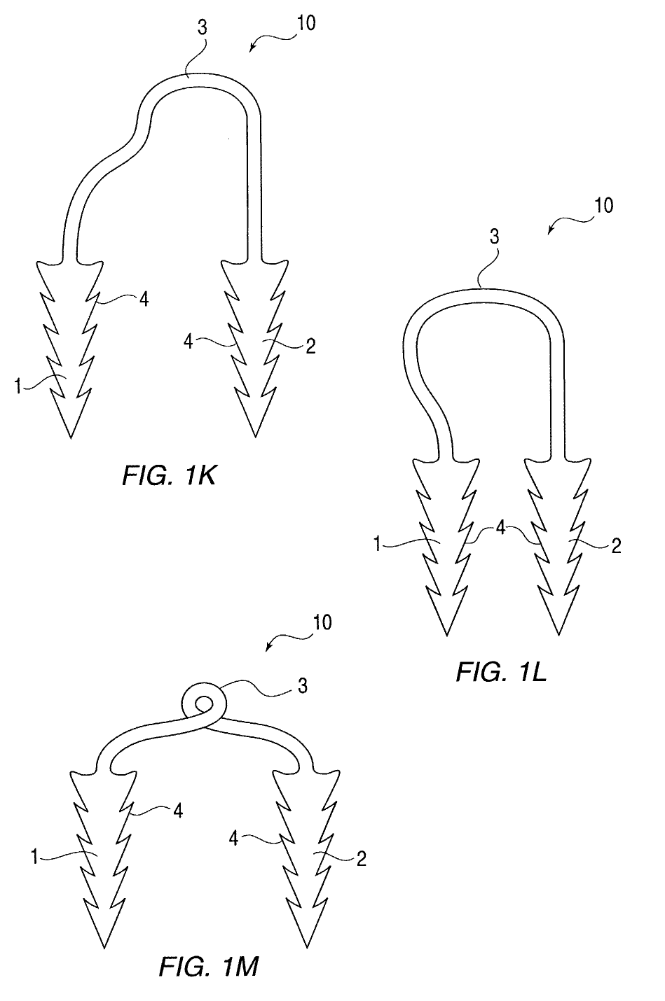

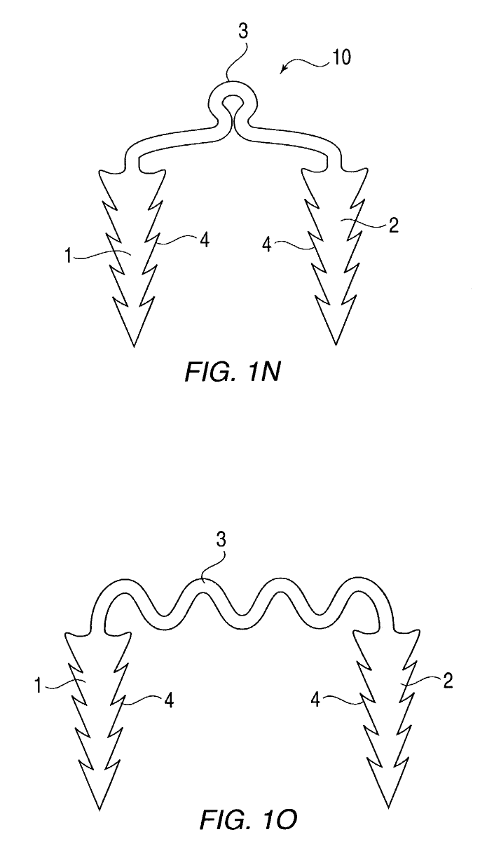

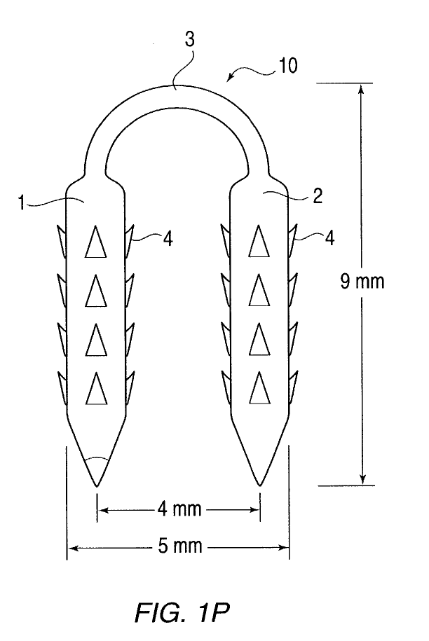

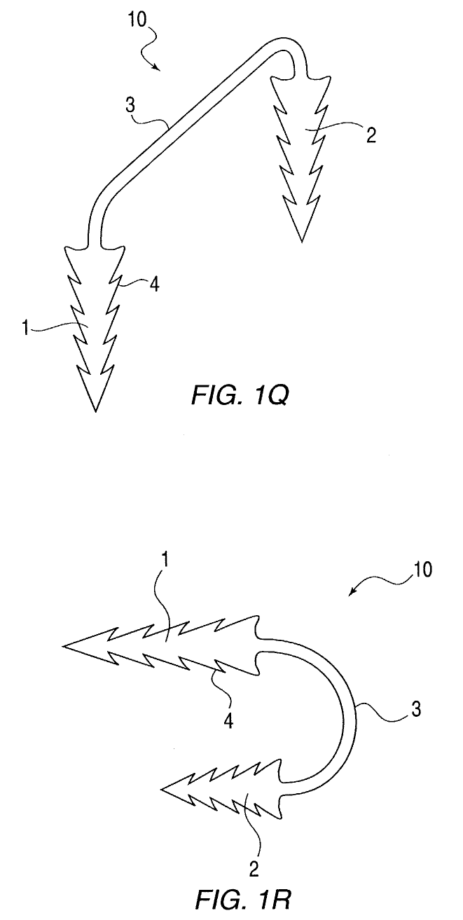

FIGS. 1A-R show views of a variety of embodiments of the fastener 10 of the present invention. The fastener 10 of the present invention may include two longitudinal implantation members 1 and 2 connected to one another by a connecting member 3. Each implantation member has a distal and proximal end.

For example, FIG. 1A shows implantation members 1 and 2 and connecting member 3. The implantation members 1 and 2 may include protuberances or protrusions 4 between a portions of the implantation members' distal and proximal ends. The protrusions 4 may be barbs, ridges, pyramids, scales, threads, serrations, or combinations thereof, running transverse or longitudinally along the implantation members' 1 and 2 surface. It is evident that other types of distal protrusions 4, than those described in the Figures may be used in the fasteners 10 of the present invention. Such protrusions are described, e.g. in co-pending U.S. patent application Ser. No. 08/887,130. The implantation members' distal ends may have sharp tips 1′ and 2′. The connecting member 3 may have a middle (horizontal) portion 3′ and curved ends 3″ and 3′″.

In a preferred embodiment of the present invention the size of a fastener may be about 9 mm from the top of the connecting member 3 to the pointed tips 1′ and 2′ of the implantation members 1 and 2, and the pointed tips 1′ and 2′ form approximately a 60° angle. The width of the connecting member may be approximately 0.5 mm and the width of the implantation members may be approximately 1.0 mm. The total width of the fastener may be approximately 2-10 mm, but preferably 4 mm from point 1′ to point 2′ and 5 mm from outer edge to outer edge.

The protrusions 4 prevent an installed fastener 10 from slipping out of a tissue portion in the proximal direction, which is opposite to the direction of installation. At least one or more of the protrusions 4 must penetrate a rupture plane inside of the tissue, in order to lock the distal portion of the fastener 10 into the tissue distally of the tear. The tapered, sharp form of the tips 1′ and 2′ of the implantation members 1 and 2 allows easy, minimally traumatic penetration of the implantation members 1 and 2 into the tissue. The protrusions 4 of the implantation members 1 and 2 allow the fastener 10 to lock the tissue when the fastener 10 is installed by pushing, shooting or hammering.

The connecting member 3 may be used to stop the fastener 10 inside of tissue, such that part of the connecting member 3 remains on the surface of the tissue during the final stage of installation. For example, if a fastener 10 of the present invention was inserted into a meniscus tissue, a portion of the connecting member 3 might be located at the bottom of a small notch on the surface of the meniscus, thus causing no disturbance to the opposite joint cartilage surface of the distal joint surface of the femur. Only a small suture loop like part 3a (not shown) of the fastener remains on the tissue surface. Therefore the combination of the implantation members 1 and 2 with protrusions 4 and the connecting member 3 lock the fastener 10 effectively in the tissue to close and fix the rupture and enhance healing.

Both the protrusions 4 of the implantation members 1 and 2 and the proximal direction of inserting the implantation members 1 and 2, together act to exert an advantageous compression to a ruptured surface when the fastener 10 is shot into a tissue and across a rupture. This compression serves to close the rupture and promote healing.

As shown in FIGS. 1A-P the fastener 10 of the present invention may have different geometries. For example, the implantation members 1 and 2 may have a cylindrical body and tapered tips, as in FIG. 1A or the implantation members 1 and 2 may have conical bodies, as in FIG. 1D. The barbs 4 may be only on one side of the implantation member, as in FIGS. 1B-1C, on two sides, as in FIG. 1A or on more than two sides. The tip(s) 1′ and 2′ of the implantation members 1 and 2 may be conical (e.g. FIG. 1A), pyramidal or non-symmetrical (e.g. FIG. 1C). The middle (horizontal) portion of the connecting member 3 may be straight (like in FIGS. 1A-1D) or curved (like in FIGS. 1E-1F).

The connecting member may also have other geometries, for example, the connecting member 3 may be bent in different ways, as is seen in FIGS. 1G-1R. Alternatively, the longitudinal axes of the implantation members may form different angles in relation to the longitudinal axis of the connecting member (as in FIG. 1Q). This type of fastener may be advantageous when the fastener is pushed into an oblique tissue surface. In another embodiment of the present invention, the implantation members may have different lengths (as in FIG. 1R). Such a fastener may be pushed in a vertical position against an oblique tissue surface such that the tips of the implantation members may touch the tissue surface simultaneously to avoid harmful tissue movements during fastener installation.

It would of course be within the skill of one of ordinary skill in the art to combine different geometries of implantation members and connecting members with each other in other ways than those shown in FIGS. 1A-R.

The fastener may be formed from a single, at least partially longitudinally drawn and oriented billet. The structure of the connecting member 3 may be drawn and oriented in the direction of its long axis to increase its strength and ductility and to reduce its diameter at least in one direction. The drawing and orientation is preferably achieved by solid state drawing of the connecting member. Also the implantation members 1 and 2 may be drawn and oriented in the direction of their long axes to increase their strength and ductility. In addition, the thickness of the connecting member 3 is smaller in at least in one direction than the thickness of the implantation members 1 and 2. In a preferred embodiment, the draw ratio is larger for the connecting member 3 than the implantation members 1 and 2. For example at least a portion of the connecting member 3 has a smaller diameter then a portion of either implantation member 1 and 2.

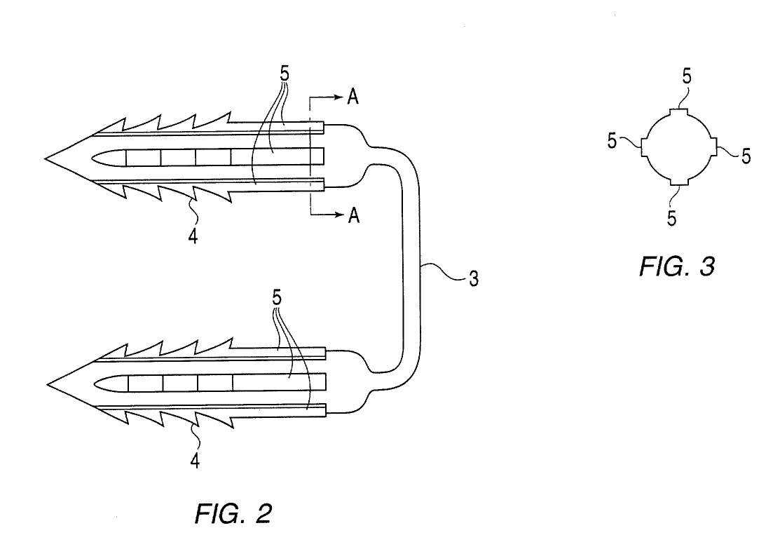

FIG. 2 shows a view of a fastener 10 where the implantation members 1 and 2 include longitudinal ridges 5. FIG. 3 shows a cross-section of implantation member 1 along line A-A. The longitudinal ridges 5 shown are advantageous for promoting healing of a rupture by providing channels that act as capillaries along the interiors of the ridges through which beneficial blood can flow along the length of the device. These channels 5 may be about 0.05-0.5 mm wide and deep, transporting blood from the highly vascularized distal portion of the tissue to the poorly vascularized proximal portion of the tissue. The distal protrusions 4 (like barbs) can be machined effectively into the longitudinal ridges 5. Further, the ridges 5 may help to guide the fastener 10 through the cannula of an installation instrument and into the soft tissue during installation.

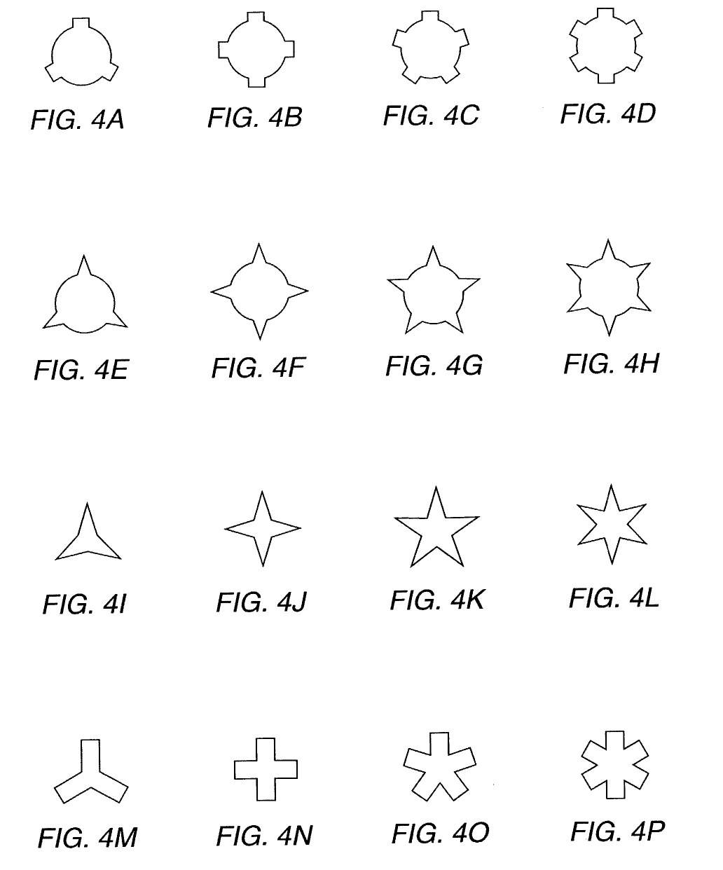

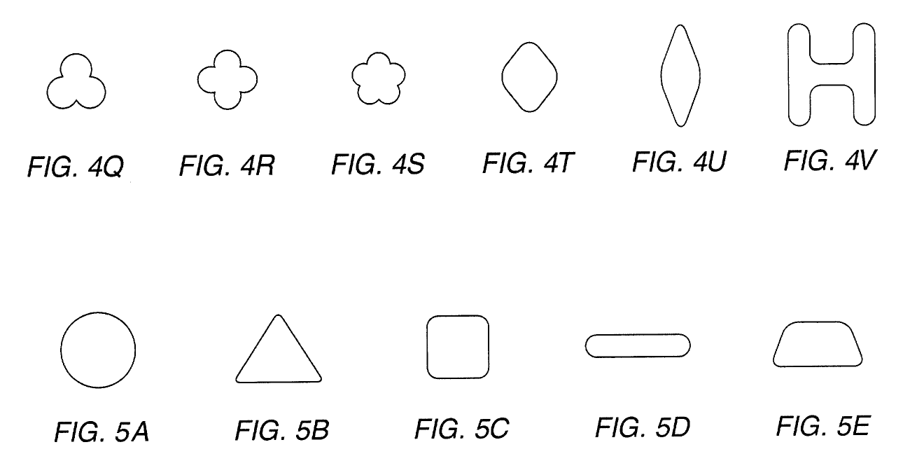

FIGS. 4A-V show examples of possible cross-sections of implantation members 1 or 2, seen from the proximal end of the member. FIGS. 5A-E show possible cross-section geometries for the connecting member 3. The implantation members 1 and 2 and the connecting member 3 may have different cross-sectional geometries from one another.

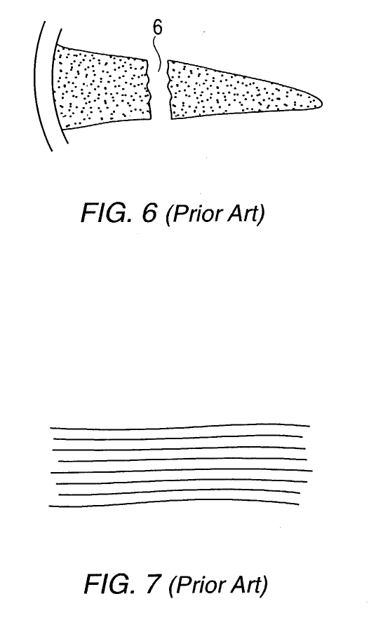

In one embodiment of the present invention, the fastener 10 is used to repair a tear in the meniscus of the knee. FIGS. 6 and 7 show the typical microstructure of a meniscus, which contains reinforcing collagen fibers. Inside meniscus tissue, collagen fibers are oriented in a horizontal plane nearly parallel to the lower surface of the meniscus. If the horizontal collagen fibers are examined in a cross-section cut of a meniscus (as shown in FIG. 6) their cut ends may be seen microscopically as points on the cross-sectional surface. The typical vertical meniscus-lesion or rupture 6 develops along the long axes of collagen fibers, because the binding forces between collagen fibers are weaker than along the long axis of the fibers.

If the internal collagen fiber structure of a meniscus is examined from the direction of the long axis of the fastener 10, i.e., from the direction from which the fastener may enter the meniscus, the collagen fibers are seen as parallel, horizontal fiber bundles, as shown in FIG. 7.

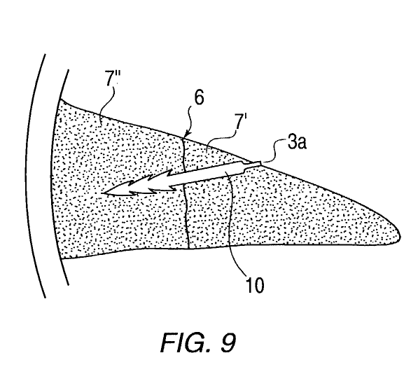

Because of the special arrangement of the main portion of the reinforcing horizontal collagen fibers inside of the meniscus, shown schematically in FIGS. 6 and 7, it is advantageous that the protrusions 4 of implantation members 1 and 2 be located at least on their upper and/or lower surfaces, so that as the fastener 10 penetrates into the meniscal tissue, the distal protrusions 4 slide forward through the collagen fiber bundles and grab finally between the horizontal collagen fiber bundles, locking the fastener 10 in place. This is shown schematically in FIG. 9.

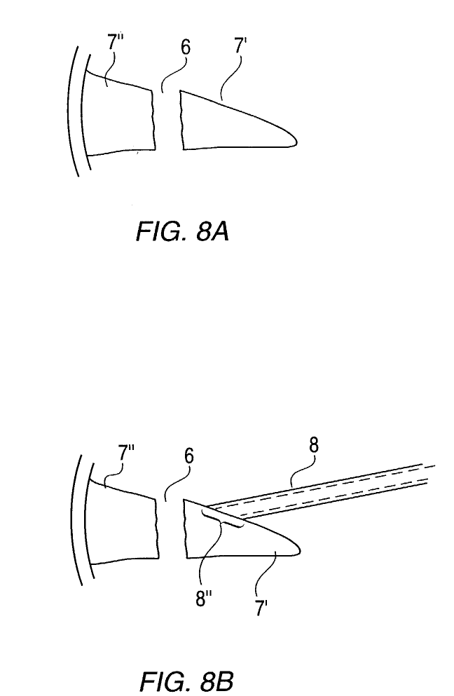

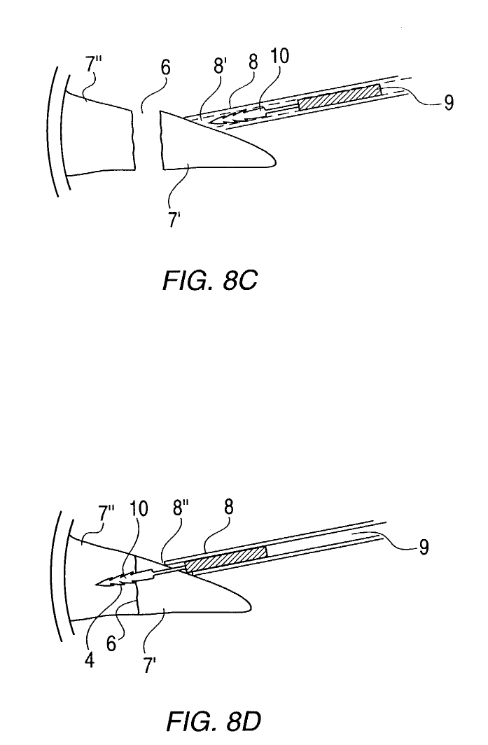

The installation of the fastener as shown in FIGS. 8A-D, results in the compression of the rupture 6 surface of a meniscus by connecting member 3 (not shown) pushing the proximal side of the rupture 7′ against the distal side of the rupture 7″ during the final phase of installation.

Because the fastener 10 is located primarily inside of the meniscus, leaving only a small, suture loop like prominence on the meniscus surface 3a, the risks of prior art devices, regarding the complications originating: (a) from the presence of the bulky proximal part of the device on the meniscal surface; or (b) from the cutting of collagen fibers inside of meniscus by the first (proximal) protrusions, are eliminated.

FIG. 8A shows as side view of a meniscus rupture 6, separating the meniscus into a proximal side, 7′, and a distal side, 7″. As seen in FIG. 8B, during installation of the fastener 10, the tip 8″ of an installation cannula 8 is pushed into the knee joint through a small incision and the tip 8″ is located on the surface of the proximal part of the meniscus 7′ in relation to the rupture 6. Insertion member 9 (not shown) within cannula 8 substantially shoots or pushes the fastener 10 (not shown) from behind into the tissue.

As seen in FIG. 8C, piston 9 moves to the left (distally) and pushes the fastener 10 through the hole 8′ inside of cannula 8. The piston 9 can be accelerated to a high speed so that the piston 9 pushes or shoots the fastener 10 with high speed into the meniscus as is shown in FIG. 8D. The piston 9 stops at the final stage of its movement by way of a stopper (not shown) at the proximal end of the piston 9, so that the tip of the piston 9 protrudes partially out of the tip 8″ of cannula 8 for about 0.5-1 mm. This pushes the fastener 10 inside of the meniscal tissue so that part of the proximal connecting member 3 of the fastener is located at the bottom of a small notch formed on the surface of the meniscus. When the location of the cannula tip 8″ on the meniscal surface is selected in a proper way, typically about 2-4 mm in front of the meniscal tear 6, and the direction of the cannula 8 is proper, the fastener 10 penetrates the proximal meniscus part 7′ and the tear plane 6 to close the tear with the compression force created by the installation push.

According to FIG. 8D, the piston 9 pushes and forces the fastener 10 inside of the meniscal tissue so that the connecting member 3 is left partially on the meniscal surface into a small notch. As soon as the piston 9 stops, typically about 0.5-1 mm below the surface of the meniscus, the connecting member 3 stops the fastener 10 and prevents its further movement into the meniscal tissue. The distal portion of the device is also pushed partially across the rupture 6 and into the distal side of the meniscus 7″, where the distal protrusions 4 prevent the slipping of the fastener 10 back in the direction opposite to the installation direction. Accordingly, the rupture 6 is closed effectively, the fastener 10 is locked in its position to keep the rupture 6 closed and only a small, suture loop-like part of the whole fastener 10 is left on the meniscal tissue.

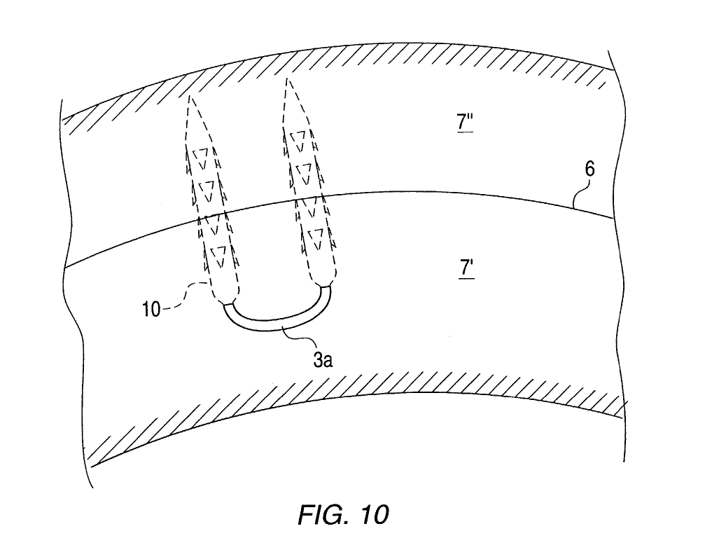

FIG. 10 shows fastener 10 of FIG. 9 as seen from the proximal direction on the surface of the meniscus. Only a small, thin suture loop-like end 3a of the fastener 10 is seen on the surface of the proximal side 7′ of the meniscus. In an advantageous embodiment, the proximal end 3a of the fastener 10 is located at the bottom of a small notch on the meniscal surface.

It is well known that the meniscus also includes oriented fibers that are not horizontal. For example, the meniscus may also contain fibers having radial or oblique orientations. The collagen fibers essentially form a three-dimensional network in the meniscus.

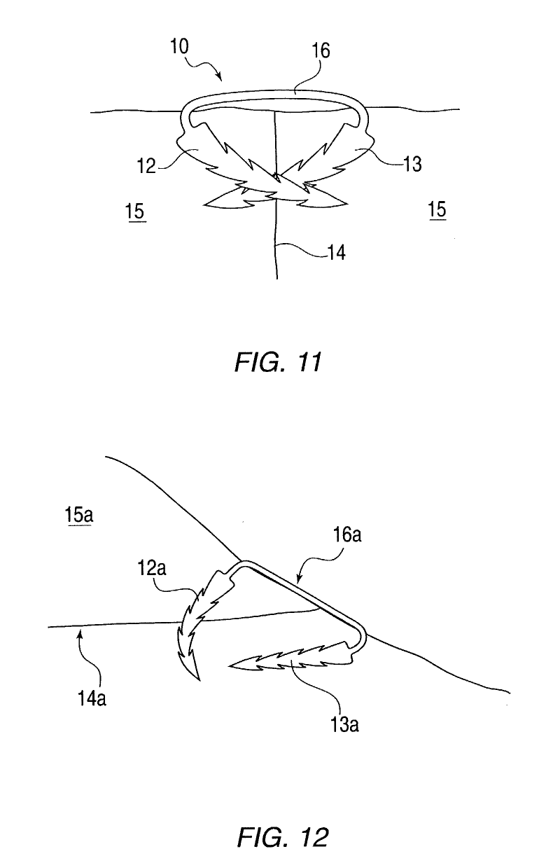

FIG. 11 shows a side view of a fastener of the present invention with curved implantation members 12 and 13, applied to close a wound 14 in a tissue 15. The implantation members 12 and 13 penetrate the wound 14 plane and cross each other while the horizontal part 16 of the connecting member remains on the surface of the tissue 15.

FIG. 12 shows a cross-sectional view of a fastener 10, which may be applied to close a horizontal rupture 14a of a meniscus 15a so that one implantation member 12a traverses the rupture plane 14a closing the rupture. Of course, it is also possible that the other implantation member 13a traverses the rupture plane as well.

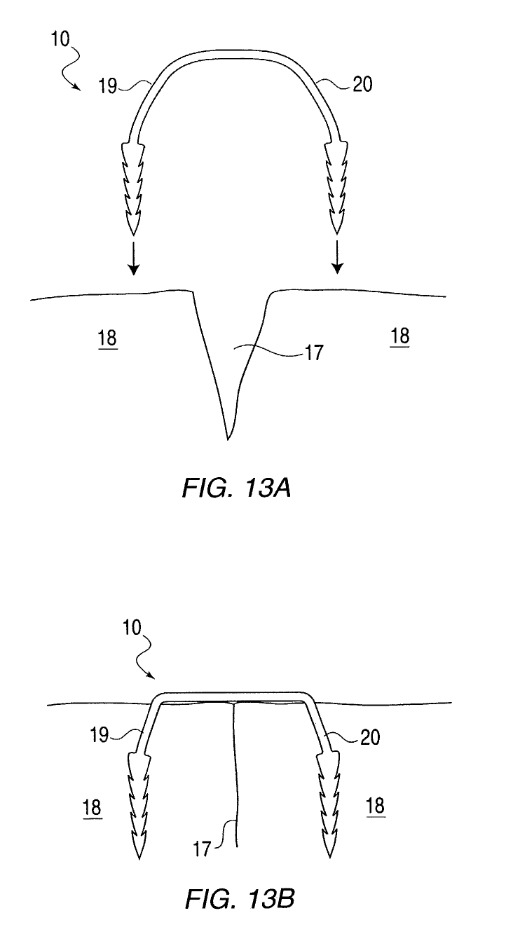

FIGS. 13A-B show a fastener, which may be used to close a wound 17 in tissue 18. Because of the tapering parts 19 and 20 of the connecting member, the wound 17 may be closed and compressed when the fastener 10 of the invention is used.

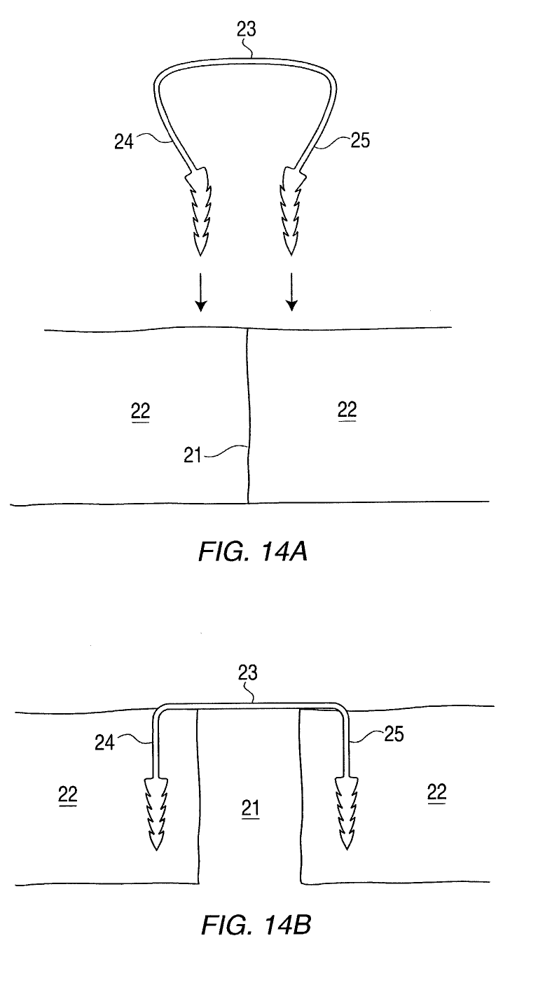

The fastener 10 of the present invention can also be applied to keep a wound open as shown in FIGS. 14A-B. Here a wound 21 in a tissue 22 is opened with a fastener 23, which has widening parts 24 and 25 in its connecting member. The opened wound 21′ of FIG. 14B may be filled, for example, with a tissue transplant to expand the tissue 22.

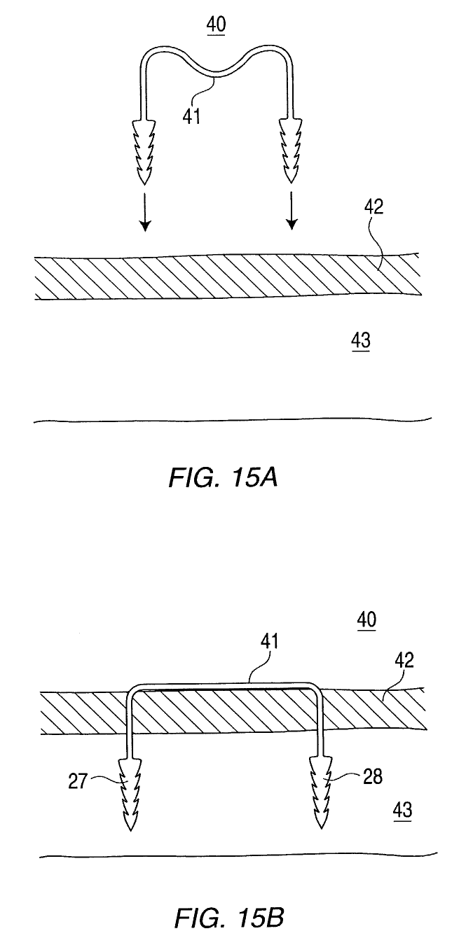

According to an advantageous embodiment of the invention a fastener 40 of the present invention may be applied to compress tissues against each other in the direction of long axes of the implantation members, as is seen in FIGS. 15A-B. When the fastener 40 with the downwards curved connecting member 41 is pushed through the first tissue 42 into the second tissue 43, which is below and in contact with the first tissue 42, the curved part of the connecting member 41 is straightened on the surface of the first tissue 42. An upwards pulling force, which compresses the boundary between tissues 42 and 43 is created in the barbed distal parts of the implantation members 27 and 28.

The fasteners of the present invention may be applied as suture anchors by knotting suture(s) into the connecting member of the fastener. The connecting member may also contain special element(s) like hole(s) for suture fixation. In addition the fasteners of the present invention may be used in securing tears or closing wounds in living tissues, these fasteners may be applied also for fixation of synthetic fibrous implants, like membranes, meshes, non-woven felts, fibrous scaffolds, etc. on or in living tissues. Such synthetic fibrous implants are described e.g. in EPO Pat. No. 0423155, U.S. Pat. No. 6,007,580 and PCT/EP 98/03030.

Further, the implant of the present invention may be used to affix another implant, like a hernia mesh, to or in a tissue. The implant may be manufactured of a polymer or a polymeric compound which is substantially bioabsorbable in tissue conditions and contains oriented reinforcing structure or the like of a polymer or polymeric compound or ceramic compound, such as bioactive glass or tricalcium phosphate.

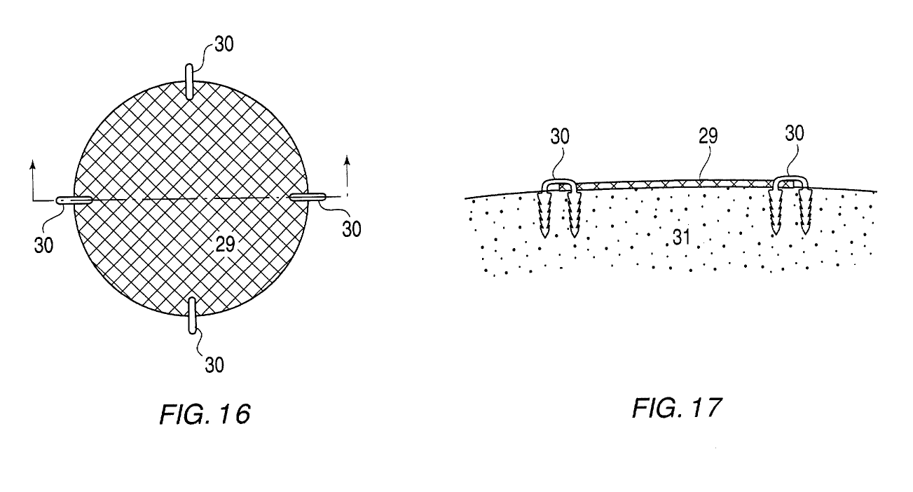

When using the fasteners of this invention in fixation of synthetic fibrous implant or biological transplant on or into living tissue, the implant or transplant is first aligned on the surface or inside of the living tissue. Thereafter fasteners are pushed (shot) one after another through the implant or transplant so that the distal barbed parts of the implantation members lock the fastener into living tissue below the implant or transplant and the suture-loop like part of connecting member remains on the implant or transplant securing it on the surface (or inside) of the living tissue. FIG. 16 shows, as seen from above, and FIG. 17 as a side view, in plane B-B of FIG. 16, how a fibrous mesh 29 has been secured with fasteners 30 on living tissue 31.

Typical living tissue transplants, which may be fixed with the fasteners of this invention are autografts, allografts and xenografts, like collagen membranes and felts, periosteum transplants or connective tissue transplants.

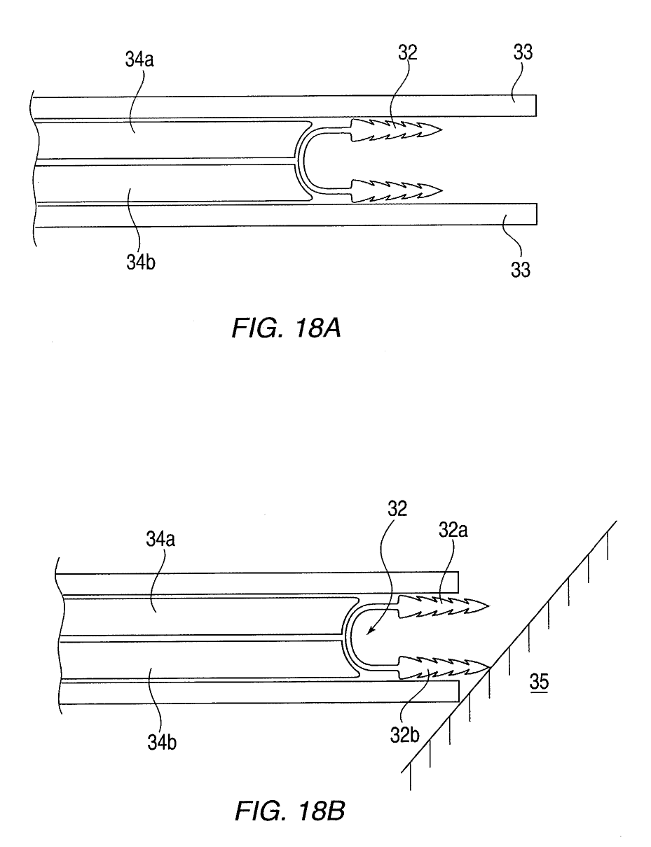

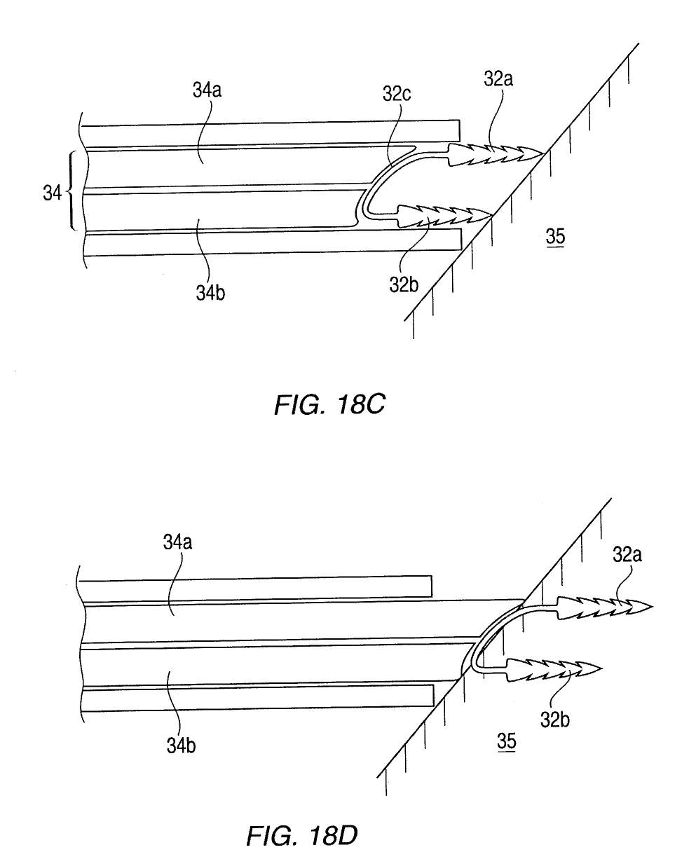

FIGS. 18A-D show an embodiment of an installation tool of the present invention, including a two-part piston and a method of using such an installation tool. This embodiment of an installation tool may be used to advantageously adjust the shape of a fastener of the present invention, so that the fastener precisely fits the tissue in which it is inserted. This fitting of the fastener may be achieved by using a two-part piston installation instrument as shown.

FIG. 18A shows a fastener 32, which may be pushed forward (to the right) inside of a cannula 33 by means of a two-part piston 34, 34a and 34b. FIG. 18B shows the “lower” implantation member 32b touching the surface of an oblique tissue 35. In order to shape the fastener 32, such that both implantation members 32a and 32b enter the tissue 35 at approximately the same time, the upper part of the piston 34a may be moved forward or ahead of 34b. This movement forces the tip of the “upper” implantation member 32a against the surface of the tissue 35 by changing the shape of the connecting member 32c of the fastener 32. The change in shape may be caused by the force applied to the fastener 32 by either part of the piston 34a or 34b. As is shown in FIG. 18C, the fastener 32 has now changed shape such that implantation member 32a is longer or extends farther than does implantation member 32b. The resulting shape of the fastener may depend on the wound to be healed. Once the fastener 32 has been adequately adjusted, both parts of piston 34 are used to apply force to the fastener 32 to insert the fastener 32 into tissue 35. As may be seen from FIG. 18D, both implantation members 32a and 32b are inserted into the tissue approximately the same distance and connecting member 32c is left on the surface of tissue 35.

Although this preferred example of an installation tool has been described, one of ordinary skill in the art would recognize that many different installation tools may be used to insert a fastener of the present invention. Therefore, the scope of the present invention is not intended to be limited.

When manufacturing a fastener of the present invention, a billet of bioabsorbable material is extruded, then cut, and then formed into the shape of a fastener. Prior to or after cutting, the entire billet or a portion thereof may be drawn.

In a preferred embodiment of the present invention, the portion of the billet to become the connecting member may be drawn to a draw ratio of about 2-15 at a temperature T, where depending on the crystallility of the billet material, T is Tₘ>T>T_{g} if the material is crystalline or T>T_{g} if the material is amorphous (Tₘ being the melting temperature of the material and T_{g} being the glass transition temperature of the material).

In another preferred embodiment of the present invention, the portion of the billet to become the implantation members may be drawn to a draw ratio of about 1.5-10 at a temperature T, where depending on the crystallility of the billet material, T is Tₘ>T>T_{g} if the material is crystalline or T>T_{g} if the material is amorphous (Tₘ being the melting temperature of the material and T_{g} being the glass transition temperature of the material).

In another preferred embodiment of the present invention, a portion of the billet may be relaxed to a lower draw ratio by heating the portion to a temperature T, where depending on the crystallility of the billet material, T is Tₘ>T>T_{g} if the material is crystalline or T>T_{g} if the material is amorphous (Tₘ being the melting temperature of the material and T_{g} being the glass transition temperature of the material).

The bioabsorbable implants of this invention may be manufactured of bioabsorbable polymers, copolymers or polymer mixtures or alloys with melt molding methods known in the prior art. It is also possible to use the techniques of U.S. Pat. No. 4,743,257 to mold in a compression or injection mold absorbable fibers and binding polymer together to create a fiber-reinforced or a self-reinforced structure. The implants of this invention may be molded in a single compression molding cycle, or the protrusions may be machined on the surface of a fastener after the molding cycle.

The oriented and/or self-reinforced structure may also be created during extrusion or injection molding of absorbable polymeric melt trough a suitable die or into a suitable mold at high speed and pressure. When cooling occurs at suitable conditions, the flow orientation of the melt remains in the solid material as an oriented or self-reinforcing structure. In an advantageous embodiment, the mold may have the form of the implant, but it is also possible to manufacture the implants of the invention by machining (possibly using heat) and thermoforming (e.g. by bending the proximal end) of injection-molded or extruded semi-finished products.

It is advantageous to make the implants of melt-molded, solid state drawn or compressed, bioabsorbable polymeric materials, which are described e.g. in U.S. Pat. Nos. 4,968,317 or 4,898,186.

The reinforcing fibers of the implant may also be ceramic fibers, like bioabsorbable hydroxyapatite or bioactive glass or tricalcium phosphate fibers. Such bioabsorbable, ceramic fiber reinforced materials are described e.g. in European Patent Application No. 0146398 and in WO 96/21628.

The oriented and/or self-reinforced or otherwise fiber reinforced implants of this invention may be manufactured by molding the reinforcement fiber-polymer matrix to the final product in a mold, whose mold cavity has the form of the final product or the final form may be machined mechanically (possibly also using heat) on a preform, such as a melt-molded and solid-state drawn rod, as is described e.g. in U.S. Pat. No. 4,968,317.

The reinforcement elements may extend into any protrusions or ridges of the implant. The reinforcement elements may also turn spirally around the long axis of the implantation members and/or of the connecting member. Also, other different orientations of reinforcement elements in elongated samples which are familiar from composite technology may be applied to the present invention. However, a general feature of orientation and/or fiber-reinforcement or self-reinforcement of the implants of this invention is that many of the reinforcing elements are oriented in such a way that they can carry effectively the different external loads (such as tensile, bending and shear loads) that are directed to the healing rupture (for example loads to a meniscus caused by the movements of the patient's knee).

According to an advantageous embodiment of the invention, the meniscal repair implant, or a special coating layer on its surface, may contain one or more bioactive substances, such as antibiotics, chemotherapeutic substances, angiogenic growth factors, substances accelerating the healing of the wound, growth hormones and the like. Such bioactive meniscal repair implants are especially advantageous in surgical use, because they chemically contribute to the healing of the lesion in addition to providing mechanical support.

The oriented and/or reinforced materials of the implants typically have initial tensile strengths of about 100-2000 MPa, bending strengths of about 100-600 MPa and shear strengths of about 80-400 MPa. Additionally, they can be made stiff, tough, and/or flexible. These mechanical properties are superior to those of non-reinforced absorbable polymers which typically show strengths between 40 and 100 MPa and may additionally be brittle (see e.g. Ref. 3 S. Vainionp\ddot{aa}, P. Rokkanen and P. Törmälä, “Surgical Applications of Biodegradable Polymers in Human Tissues”, Progr. Polym. Sci 14/1989, pp. 679-716).

A special advantage of the present invention is that there is no bulky proximal end in these fasteners. They can be made relatively thin e.g. with implantation member diameters about 1-2 mm and connecting member diameters about 0.2-1 mm with a part of the connecting member resembling a minimally traumatic suture loop on the meniscal surface.

The implants of the present invention may be sterilized by any of the well known sterilization techniques, depending on the type of material used in manufacture of the implant. Suitable sterilization techniques include heat or steam sterilization, radiation sterilization such as cobalt 60 irradiation or electron beams, ethylene oxide sterilization, and the like.

After the description above of the present invention and certain specific embodiments thereof, it will be readily apparent to those skilled in the art that many variations and modifications may be made to the present invention without departing from the spirit and scope thereof.

The principles of the present invention described broadly above will now describe with reference to the following specific example, without intending to restrict the scope of the present invention.

### EXAMPLE 1

A cylindrical, continuous billet with a thickness of about 1.5 mm was extruded from PLA 96L/4D polymer (i.v.≈6.5, manufacturer: Purac Biochem B.V., Holland) with a single screw extruder (Extrudex, φ 15 mm). The billet was drawn in the solid state (at temperature of 105-110° C.) to a draw ratio of 6. The drawn billet was cut into pieces of the length of 60 mm. The cut sample was moved into a straight, cylindrical mold with a middle cavity length of 6 mm and a diameter of 0.5 mm and with outer cavity parts with a length of about 2×25 mm and a diameter of about 1.0-1.1 mm. The implantation member parts were partially relaxed in the outer cavity parts to the draw ratio of about 2.5 by heating the outer cavity parts to the temperature of 85° C. for 30 seconds. During relaxation the implantation member parts shortened and thickened to the diameter of about 1.0-1.1 mm. The partially relaxed sample was removed from the mold. The tips of the implantation members were sharpened and barbs were cut on three sides of the implantation members. Finally the sample was bent into the shape of a staple. The staple with its dimensions is shown in FIG. 1P.

The staples were tested biomechanically using porcine meniscus. The staples were implanted into the menisci using arthroscopic prototype instrument, which consisted of a flat cannula part and a pusher part. The curved connecting part of the staple fitted firmly against the curved tip of the pusher. The staple slid through the cannula freely during implantation without the need to fix or join it with the pusher in any way.

After implantation the staples were pulled out of the menisci using a hook-type steel device and the maximum force was registered. Measured pullout forces varied about from 53 to 96 N in six test specimens. These values were significantly higher than the average load to failure of prior art staples (25.33±14.66N in T. D. Koukoubis et al., Knee Surg. Sports Traumatol, Arhroscopy, 5 (1997) 25-30).

We claim:

1. A fastener for tissue repair, comprising:
a first longitudinal member having distal and proximal ends, and transverse protrusions;
a second longitudinal member having distal and proximal ends, and transverse protrusions; and
a connecting member connecting the proximal end of the first member to the proximal end of the second member;
wherein at least a portion of the connecting member has a smaller diameter than a portion of either the first member or the second member.

2. The fastener of claim 1, wherein the connecting member is longitudinally drawn and oriented.

3. The fastener of claim 1, wherein the first member or the second member is longitudinally drawn and oriented.

4. The fastener of claim 1, wherein the first member and the second member are longitudinally drawn and oriented.

5. The fastener of claim 1, wherein the protrusions are selected from the group consisting of ridges, barbs, pyramids, threads, scales, serrations, or combinations thereof.

6. The fastener of claim 1, further comprising at least one longitudinal ridge located along the first member, the second member, or both the first and second members.

7. The fastener of claim 6, wherein the at least one ridge is located between the distal and the proximal ends of each member.

8. The fastener of claim 7, wherein the protrusions protrude from the at least one longitudinal ridge along each member.

9. The fastener of claim 1, wherein the fastener is a tissue staple.

10. The fastener of claim 1, wherein the fastener comprises bioactive material.

11. The fastener of claim 1, wherein the fastener comprises fiber reinforcements.

12. The fastener of claim 1, wherein the fastener comprises bioactive substances.

13. A method for repairing tissue using the fastener of claim 1, comprising:
pushing the distal portions of the first and second longitudinal members of the fastener into the tissue; and
partially embedding the fastener in the tissue, wherein at least a portion of the connecting member is visible on the surface of the tissue.

14. The method of claim 13, further comprising attaching a suture to the connecting member.

15. The method of claim 13, wherein the pushing is done by an insertion tool.

16. The method of claim 15, wherein the insertion tool includes a piston having two independently movable parts.

17. The method of claim 16, wherein one of the piston's independently movable parts forces the fastener to change shape prior to pushing.

18. A method of fixating an implant to tissue using the fastener of claim 1, comprising:
placing the implant on the tissue;
pushing the distal portions of the first and second members of the fastener through the implant into the tissue; and
embedding the fastener into the tissue, wherein at least a portion of the connecting member of the fastener is visible on a surface of the implant.

19. The method of claim 18, wherein the implant is selected from the group consisting of, synthetic polymeric mesh, collagenous mesh, periosteum transplant, or a transplant including connective tissue.

20. A kit comprising:
the fastener of claim 1; and
an insertion tool, comprising a cannula, a piston, and a tip.

21. The kit of claim 20, wherein the fastener is loaded in the insertion tool.

22. The kit of claim 20, wherein the insertion tool further includes a stopper.

23. A method of using the kit of claim 20, comprising:
loading a fastener into an insertion tool, wherein the distal portions of the first member and the second member are proximal to the insertion tool tip;
pressing the tip of the insertion tool against a tissue;
pushing the fastener into the tissue through the tip of the insertion tool by accelerating the piston; and
stopping the insertion of the fastener into the tissue through a stopper, wherein at least a portion of the connecting member of the fastener is visible on a surface of the tissue.

24. A method of manufacturing a fastener of claim 1, comprising:
extruding a billet of bioabsorbable material;
cutting the billet; and
bending the cut billet into the form of the fastener of claim 1.

25. The method of claim 24, wherein a portion of the billet, which is to become the connecting member, is drawn to a draw ratio between 2-15 at a temperature T, wherein Tₘ>T>T_{g}, and Tₘ is the melting temperature of the material and T_{g} is the glass transition temperature.

26. The method of claim 25, wherein drawing of the portion to become the connecting member is done prior to cutting the billet.

27. The method of claim 25, wherein drawing of the portion to become the connecting member is done after cutting the billet.

28. The method of claim 24, wherein a portion of the billet, which is to become the connecting member, is drawn to a draw ratio between 2-15 at a temperature T, wherein T>T_{g}, and T_{g} is the glass transition temperature.

29. The method of claim 28, wherein drawing of the portion to become the connecting member is done prior to cutting the billet.

30. The method of claim 28, wherein drawing of the portion to become the connecting member is done after cutting the billet.

31. The method of claim 24, wherein a portion of the billet, which is to become the first and second members, is drawn to a draw ratio between 1.5-10 at a temperature T, wherein Tₘ>T>T_{g}, and Tₘ is the melting temperature of the material and T_{g} is the glass transition temperature.

32. The method of claim 31, wherein drawing of the portion to become the first and second members is done prior to cutting the billet.

33. The method of claim 31, wherein drawing of the portion to become the first and second members is done after cutting the billet.

34. The method of claim 24, wherein a portion of the billet, which is to become the first and second members, is drawn to a draw ratio between 1.5-10 at a temperature T, wherein T>T_{g}, and T_{g} is the glass transition temperature.

35. The method of claim 34, wherein drawing of the portion to become the first and second members is done prior to cutting the billet.

36. The method of claim 34, wherein drawing of the portion to become the first and second members is done after cutting the billet.

37. The method of claim 24, wherein the first and second members are sharpened.

38. The method of claim 24, wherein protuberances are formed on a surface of the first and second members.

39. The method of claim 24, 25, 28, 31, 34, 37, or 38, wherein a portion of a drawn billet is relaxed to a lower draw ratio by heating it to a temperature T, wherein Tₘ>T>T_{g}, and Tₘ is the melting temperature of the material and T_{g} is the glass transition temperature of the material.

40. The method of claim 24, 25, 28, 31, 34, 37, or 38, wherein a portion of a drawn billet is relaxed to a lower draw ratio by heating it to a temperature T, wherein T>T_{g}, and T_{g} is the glass transition temperature of the material.
